(19) United States
(12) Plant Patent Application Publication
(10) Pub. No.: US 2007/0011790 P1
(43) Pub. Date: Jan. 11, 2007

(54) IMPATIENS PLANT NAMED 'CAMEO BRIGHT SCARLET'

(50) IMPATIENS WALLERIANA; IMPATIENS WALLERIANA

(76) Inventor: Xenia Cascante, Alajuela (CR)

Correspondence Address: THE WEBB LAW FIRM, P.C., 700 KOPPERS BUILDING, 436 SEVENTH AVENUE, PITTSBURGH, PA, 15219, US

(21) Appl. No.: 11/177,535

(22) Filed: Jul. 8, 2005

(51) Int. Cl. A01H 5/00

(52) U.S. Cl. PLT/317

(57) ABSTRACT

‘Cameo Bright Scarlet’ is a new variety of impatiens that exhibits full, double scarlet colored flowers that face upward.

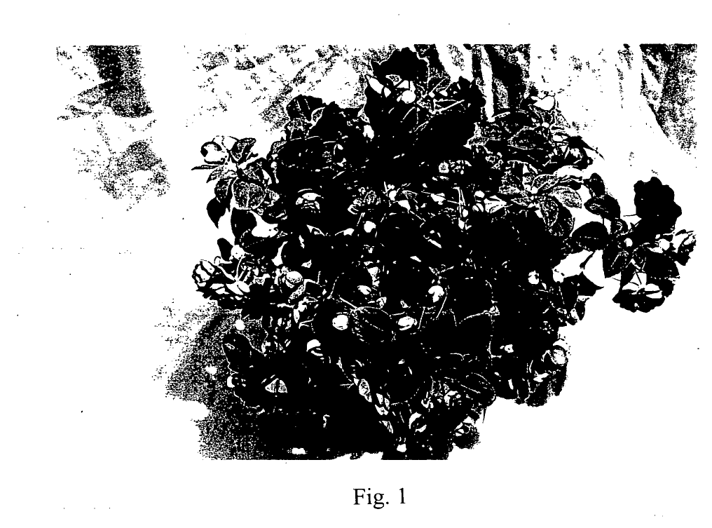

### BOTANICAL CLASSIFICATION

Impatiens wallerana

### VARIETAL DENOMINATION

‘Cameo Bright Scarlet’

### BACKGROUND OF THE INVENTION

The present invention comprises a new and distinct cultivar of Impatiens wallerana known by the varietal name ‘Cameo Bright Scarlet’ (breeder reference number IM12109). The new variety was discovered in Quebradilla, Cartago, Costa Rica. The new variety was developed as follows:

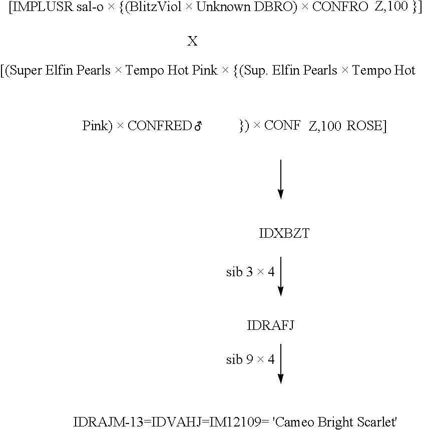

The purpose of the breeding program was to develop a fully double-flowering cultivar that is compact, mounded, having good garden performance, and that does not burn in bright sunlight. Comparisons between the new variety and its parents cannot be made, as the parental varieties no longer exist. The new variety is similar in growth rate to ‘Cameo Scarlet’ (U.S. Plant Pat. No. 12,036), but outward facing flowers. The new variety was first asexually reproduced by stem tip cuttings in Quebradilla, Cartago, Costa Rica. The new variety has been trial and field tested and has been found to retain its distinctive characteristics and remain true to type through successive propagations. Characteristics that distinguish the new variety from others known to the breeder include:
Large size flowers that are scarlet in color (45B) that do not sunburn on the underside of the flowers as they face upward;
High petal number of 35 to 40;
Compact, mounded spreading growth habit with upward facing flowers carried above the leaf canopy;
Self branching if propagated from a vegetative cutting;
Distinct rounded leaf shape and somewhat larger, mature leaf that results in a dense foliaged plant;
Flowers remain full double even when the plant is subjected to environmental stress like excessive drying, low levels of fertilizer and very high temperatures;
Compact spreading growth habit under most growing conditions makes the new variety ideal for hanging baskets and large containers;
Can be finished in 20 cm to 30 cm hanging baskets in 8 to 10 weeks and 10 and 15 cm pots can be finished in 5 to 6 weeks;
Free of any major disease or insect problems; and
The new variety can be grown at moderate levels of sunlight, but requires some shade outdoors in the summer.

The new variety was grown in a 15 cm container in Lompoc, Calif. under Dynaglass with 30% shade and 21-24° C. day temperatures and 16-18° C. night temperatures. The new variety initiates roots in 10 days in the summer and 12 days in the winter at 21° C. The new variety exhibits developed roots at 21 days at an average temperature of 21° C. Its rooting habit is very dense, fibrous and freely branching with fine roots.

### DESCRIPTION OF THE DRAWING

The accompanying photographic drawing illustrates the new variety, with the color being as nearly true as is possible with color illustrations of this type.

### DESCRIPTION OF THE PLANT

The following detailed description sets forth the characteristics of the new cultivar. The data which defines these characteristics were collected by asexual reproductions carried out in Lompoc, Calif. The color readings were taken indoors under 4000-5000 foot candles of light. The new variety was twelve weeks old when described. Color references are primarily to the 1966 R.H.S. Colour Chart of The Royal Horticultural Society of London.
Plant:
Form: Mounded spreading.
Height (from soil to top of flowers): 18-20 cm.
Plant diameter: 25-30 cm.
Vigor: Vigorous, but not leggy or open.
Branching habit: Highly self-branched.
Lateral branches:
   Color.—147C with most having light 184D flecking at the basal end of the stem.
   Texture.—Glabrous.
   Length.—20-22 cm.
   Diameter.—6-8 mm.
   Internode length.—10-25 mm, but variable.
   Strength.—Strong.
   Quantity.—10 or more.
Foliage:
   Arrangement.—Alternate.
   Number of leaves per stem.—16 or more.
   Size of leaf.—Length: 5-5.5 cm. Width: 2.5-3 cm.
   Shape of leaf (generally).—Ovate.
   Shape of apex.—Acute.
   Shape of base.—Acute to rounded.
   Texture.—Glabrous.
   Aspect.—Facing upward.
   Margin type.—Serrated with fine ciliate.
   Fragrance.—None.
   Color.—Young leaves: Upper surface: 147A. Lower surface: 146B. Mature leaves: Upper surface: Between 146A and 147A. Lower surface: 146B.
   Petiole.—Length: 10-12 mm. Diameter: 3 mm. Color: 147C. Texture: Glabrous.
   Veins.—Venation pattern: Pinnate. Color: Upper surface: 147B. Lower surface: 146A.
Flower:
Natural flowering season at specified locations: Year-round under greenhouse conditions; does not respond to changes in day length.
Flower type and habit: Double-flowers above leaf canopy.
Number of flowers per plant: 75-100 on mature plant.
Time of day of flower opening: Morning.
Fragrance: None.
Longevity: 5-7 days, but environment affects.
Flower height: 20 mm.
Flower diameter: 45 mm.
Bud:
   Stage of development when described.—Mature.
   Shape.—Ovate.
   Diameter.—15 mm.
   Length.—17 mm.
   Color.—44D just before opening.
Petals:
   Arrangement.—Whorls of 5.
   Quantity.—35-40.
   Length.—Banner petals: 22 mm.
   Width.—Basal Whorl: 20 mm.
   Shape.—Heart to ovate.
   Apex.—Rounded with inner petals showing indentation.
   Base.—Rounded.
   Margin.—Entire.
   Texture.—Smooth, glabrous.
   Color.—When opening: All petals (upper surfaces): 45A. All petals (lower surfaces): 41A with 155A near midrib. Fully opened: All petals (upper surfaces): 45B. All petals (lower surfaces): 41B with 41D around midrib.
Spur:
   Quantity.—1 per flower.
   Length.—35 mm.
   Diameter at apex.—less than 1 mm.
   Diameter at flower.—45 mm.
   Aspect.—Flowers face outward and do not hang.
   Color.—146C.
Peduncles:
   Length.—25 mm.
   Diameter.—2 mm.
   Color.—146D.
   Texture.—Glabrous.
   Orientation.—45° angle.
   Strength.—Strong.
Disease resistance: Resistant.
Weather tolerance: Rain tolerant with slight tolerance to wind.
Lasting quality:
   On plant.—5-7 days, but environment alters.
   Of cut flowers.—Not applicable.
Seed production and description: Not applicable.
Fruit production and description: Not applicable.
Reproductive organs: Not applicable due to full, double flowers.

I claim:

1. A new and distinct variety of impatiens named ‘Cameo Bright Scarlet’ as described and illustrated.
